SEQUENCE LISTING (4 sequences)

SEQ ID NO: 1 - DNA, 24 bp, synthetic construct
  source 1..24: mol_type = other DNA
gattaagccatgcaagtcgaacga

SEQ ID NO: 2 - DNA, 22 bp, synthetic construct
  source 1..22: mol_type = other DNA
catcgtttacggccaggactac

SEQ ID NO: 3 - DNA, 22 bp, synthetic construct
  source 1..22: mol_type = other DNA
cttctggaataagggctgggca

SEQ ID NO: 4 - DNA, 24 bp, synthetic construct
  source 1..24: mol_type = other DNA
ctcctcaaagaacccagattcgac

(19) United States
(12) Patent Application Publication
(10) Pub. No.: US 2026/0049340 A1
(43) Pub. Date: Feb. 19, 2026

(54) NOVEL METHANOTHERMOBACTER WOLFEII STRAIN AND USE IN PRODUCTION OF RENEWABLE NATURAL GAS AND CO2 SEQUESTRATION

(71) Applicant: WASHINGTON STATE UNIVERSITY, Pullman, WA (US)

(72) Inventors: Birgitte AHRING, Richland, WA (US); Fuad ALE ENRIQUEZ, Richland, WA (US)

(21) Appl. No.: 18/807,235

(22) Filed: Aug. 16, 2024

(51) Int. Cl. C12P 5/02; C12N 1/00; C12R 1/00

(52) U.S. Cl. CPC C12P 5/023; C12N 1/00; C12R 2001/00

(57) ABSTRACT

The present disclosure pertains to a Methanothermobacter wolfeii archaeal strain identified as BSEL1 and progeny and mutants thereof. The archaeal strain BSEL1 is capable of growing and producing renewable natural gas (RNG) from a supply of carbon dioxide (CO₂) and hydrogen (H₂) without the addition of organic supplements, and further to grow in wastewater with an exceptionally high growth rate. The strain is further capable of reducing CO₂ to a very low level when grown, e.g., in a trickling bed reactor. BSEL1 is of major importance for direct addition of RNG into the natural gas grid.

### STATEMENT OF FEDERALLY SPONSORED RESEARCH AND DEVELOPMENT

This invention was made with government support under grant no. DE-EE0008933 awarded by U.S. Department of Energy. The United States government has certain rights in the invention.

### SEQUENCE LISTING

This application includes as the Sequence Listing the complete contents of the accompanying XML file “12770145AA sequence listing” created Aug. 8, 2024, containing 4509 bytes, hereby incorporated by reference.

### BACKGROUND OF THE INVENTION

### Technical Field

The disclosure generally relates to a Methanothermobacter wolfeii bacterium and methods of its use to capture CO₂ and generate renewable biofuel. In particular, the disclosure provides the bacterium M. wolfeii BSEL1, an archaeon that utilizes CO₂ as an electron acceptor to produce methane (CH₄) without the use of organic supplements.

### Description of Related Art

Methanothermobacter is a group of archaea consisting of hydrogenotrophic methanogens which are thermophiles, strict anaerobes, and chemolithotrophs. Hydrogenotrophic methanogens utilize H₂ as an electron donor to convert carbon dioxide CO₂ into methane, CH₄. Methane is one of the most versatile forms of energy and can be stored easily. There already exists much infrastructure for transporting and distributing methane as well as infrastructure for converting methane into electricity and for powering vehicles. Methane also has the highest energy density per carbon atom of all fossil fuels, and therefore of all fossil fuels, methane releases the least carbon dioxide per unit energy when burned. Hence, systems for producing methane, e.g., through converting carbon dioxide and electricity into methane, would be highly useful and valuable in all energy generation and utilization industries.

The metabolism of hydrogenotrophic methanogens is based on the Wolfe cycle, an autotrophic pathway that relies on Acetyl CoA. The produced CH₄ can serve as a renewable natural gas (RNG) for various sustainable energy applications. RNG can be conveniently distributed through existing natural gas grids, enabling its use for industrial processing, general heating and electricity generation as well as drying and refrigeration processes.

Despite their autotrophic metabolism, methanogens often require the addition of organic compounds to synthesize materials essential for protein synthesis and rapid growth such as proteinogenic amino acids. These organic compounds, such as glucose, acetate, yeast extract, and/or peptone, are typically supplied in the anaerobic medium. However, the cost of these chemicals significantly contributes to the expense of cultivating methanogens, discouraging their use.

It would be advantageous to have available a methanogen capable of generating CH₄ from CO₂ which is capable of growing in minimal media, without the addition of organic compounds.

### SUMMARY OF THE INVENTION

Disclosed herein is a newly isolated variant of M. wolfeii, M. wolfeii BSEL1, that was specifically adapted in laboratory settings to advantageously achieve optimal growth and CH₄ production in mineral media without organic supplements besides vitamins (e.g., devoid of a dedicated supply of a macronutrient organic feedstock). Moreover, methods presented herein illustrate how this variant can be employed to produce renewable natural gas, thereby paving the way for more efficient and sustainable CO₂ sequestration processes and renewable energy sourcing. In the methods and systems of the present invention, one or more CO₂ waste-gas streams are combined with hydrogen and undergo a microbial fermentation process catalyzed by BSEL1, producing methane and water.

It is an object of the present disclosure to provide an engineered strain of Methanothermobacter wolfeii deposited at the American Type Culture Collection under the number PTA-127814, and progeny and mutants thereof.

Also provided is an M. wolfeii culture, comprising the engineered strain of Methanothermobacter wolfeii deposited at the American Type Culture Collection under the number PTA-127814, and an anaerobic culture medium. In some aspects, the culture medium is selected from at least one of a wastewater and a mineral media. In other aspects, the culture medium further comprises a supply of carbon dioxide gas and a supply of hydrogen gas. In yet additional aspects, the culture medium does not include organic macronutrients. And in other aspects, the culture is maintained at a pH of 8.0 to 8.5. In further aspects, the culture is maintained at a temperature of 62-66° C. In other aspects, the culture medium further comprises resazurin as a redox indicator and cysteine and/or Na₂S as a reducing agent(s). In some aspects, the culture does not comprise tungsten.

Also provided is a method of biogas production, comprising: culturing engineered strain of Methanothermobacter wolfeii deposited at the American Type Culture Collection under the number PTA-127814, with a supply of carbon dioxide gas and a supply of hydrogen gas under anaerobic conditions.

Also provided is a method of upgrading biogas to renewable natural gas, comprising: culturing an engineered strain of Methanothermobacter wolfeii deposited at the American Type Culture Collection under the number PTA-127814, with a supply of carbon dioxide gas and a supply of hydrogen gas under anaerobic conditions.

Also provided is a method of carbon sequestration, comprising: culturing an engineered strain of Methanothermobacter wolfeii deposited at the American Type Culture Collection under the number PTA-127814, with a supply of carbon dioxide gas and a supply of hydrogen gas under anaerobic conditions. In certain aspects, the carbon dioxide gas is or comprises waste CO₂.

Also provided is a method of producing methane, comprising: culturing an engineered strain of Methanothermobacter wolfeii deposited at the American Type Culture Collection under the number PTA-127814, in wastewater with a supply of carbon dioxide gas and a supply of hydrogen gas under anaerobic conditions; and recovering methane produced from the culturing step.

Also provided is a medium comprising means for converting CO₂ and H₂ to methane in a minimal media, wherein the means is the engineered strain of Methanothermobacter wolfeii deposited at the American Type Culture Collection under the number PTA-127814. In some aspects, the culture medium does not comprise tungsten.

Also provided is an engineered strain of Methanothermobacter wolfeii having a formylmethanofuran dehydrogenase F subunit (fwdF) with serine, aspartic acid and isoleucine at positions 17, 69, and 228, respectively, and a D subunit (fwdD) with valine at position 46.

### BRIEF DESCRIPTION OF THE DRAWINGS

FIG. 1. Accumulated gas consumption (AGC) within the Balch tubes during the enrichment cultivation of hydrogenotrophic archaea in a pressurized headspace of H₂ and CO₂ on BA medium anaerobic digestate was used as inoculum for generation I. The antibiotics ampicillin and vancomycin were added to generations III and IV (only generation IV is shown). Increase in the gas consumption rate in the last generation suggests adaptation of the culture in the minimal medium used for cultivation.

FIG. 2A-C. Methanothermobacter wolfeii strain BSEL1. (A) Isolated archaeal colonies growing on the surface of agar in a roll tube under a headspace of H₂ and CO₂ as energy and carbon sources, respectively. NH₄⁺ was used the sole nitrogen source. (B) Phase contrast micrograph; the isolate displays a crooked rod-shaped morphology characteristic of hydrogenotrophic methanogens. (C) Position of M. wolfeii BSEL1 in the phylogenetic tree of the Methanothermobacter genus (16S rRNA gene). The percentage of phylogenetic trees in which the associated taxa clustered together is shown above the branches. The tree is drawn to scale, with branch lengths measured as the number of substitutions per site. M. wolfeii BSEL1 shares 99% nucleotide sequence identity with the 16S rRNA gene of the type strain DSM 2970. The variants OCM36 and SIV6 were not included in the phylogenetic analysis. This was because the taxonomic information from M. wolfeii OCM36 could not be found in any public nucleotide sequences repository, and the RefSeq records of M. wolfeii SIV6 had been tagged as “anomalous”. Therefore, as an outcome of this work, M. wolfeii BSEL1 is currently the only isolated subtype of the M. wolfeii species phylogenetically identified with valid genomic data.

FIG. 3A-E. Influence of nutrients on the growth parameters of M. wolfeii BSEL1. Non-significant differences (P-value>0.05) are not displayed. (A) Effect of vitamins (V) and 0.1% YE on the specific growth rate in BA medium. Microbial growth was inhibited in the presence of YE (Conover test P-value≤0.05/2). (B) Effect of using wastewater as growth medium. No statistical significance was found (Kruskal-Wallis P-value=0.15) when cultivating M. wolfeii BSEL1 in minimal BA medium (BA), wastewater (WW), and wastewater added with vitamins (WW+V). (C-E) Effect of NH₄Cl as sole nitrogen source over the specific growth rate (μ) and lag phase duration (t lag) of M. wolfeii BSEL1. High correlation between t lag and the NH₄Cl content was detected, while u was independent from the concentration of nitrogen source.

FIGS. 4A and B. Actively growing M. wolfeii BSEL1 after 8 months of semi-continuous cultivation in BA medium. (A) Phase contrast micrograph. (B) Fluorescence micrograph targeting the coenzyme F420, an autofluorescent indicator of viable methanogenic cells. The field turned completely dark after 20 min of exposure, showing the quick decay of the coenzyme F420 after the M. wolfeii BSEL1 cells die by exposure to 02. Both micrographs display the same field at 63×.

FIG. 5. Headspace gas composition during CO₂ fermentation in a bubble column bioreactor. M. wolfeii BSEL1 was used as biocatalyst. The bioreactor was fed with a biogas:H₂ mimic (61.1% H₂, 25.8% CH₄, and 13.1% CO₂). The H₂:CO₂ molar ratio in the headspace decreased over time during the first 21 h of operation, indicating CO₂ generation from HCO₃⁻ in the mineral medium, ultimately decreasing the apparent CO₂ consumption. Before providing H₂ pulses, 8 mmol of CO₂ were produced from HCO₃⁻ during the fermentation cycle. The final gas composition after providing extra H₂ was 0.6% H₂, 98.5% CH₄, and 0.9% CO₂. The pH in the bioreactor increased from 7.0 to 8.4 during the 51 h of operation, indicating consumption of the CO₂/HCO₃⁻ buffer. The capacity of M. wolfeii BSEL1 to generate CH₄ from CO₂ during gas fermentation was thus demonstrated. Tuning of the process avoids the need of additional H₂.

FIG. 6. Circular chromosomal map of Methanothermobacter wolfeii strain BSEL1. The whole-genome sequence was annotated through NCBI PGAP. Arrows and labels represent only the genes that encode non-hypothetical proteins. Bars depict the BLAST comparison between strain BSEL1 and the type strain DSM 2970; the height of the bars is proportional to the alignment score between local nucleotide sequences.

FIGS. 7A and B. Analysis of orthologous genes in Methanothermobacter wolfeii BSEL1 and DSM 2970 strains. (A) Bar plot showing the number of genes classified in the categories of the COG. (B) KEGG pathway enrichment analysis. Bars display the abundance of translated genes classified in the KEGG functional categories.

FIG. 8. Fed-batch gas fermentation in bubble-column configuration using BSEL1 as the biocatalyst for H₂ and CO₂ conversion into CH₄.

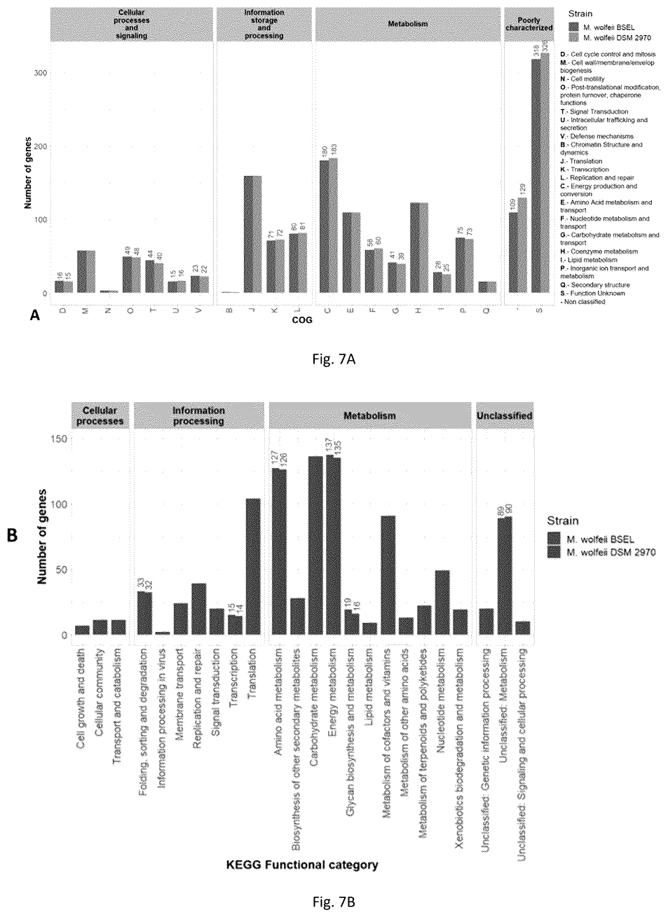

FIG. 9. Continuous gas fermentation in trickling bed configuration using BSEL1 as the biocatalyst for biogas conversion into renewable natural gas.

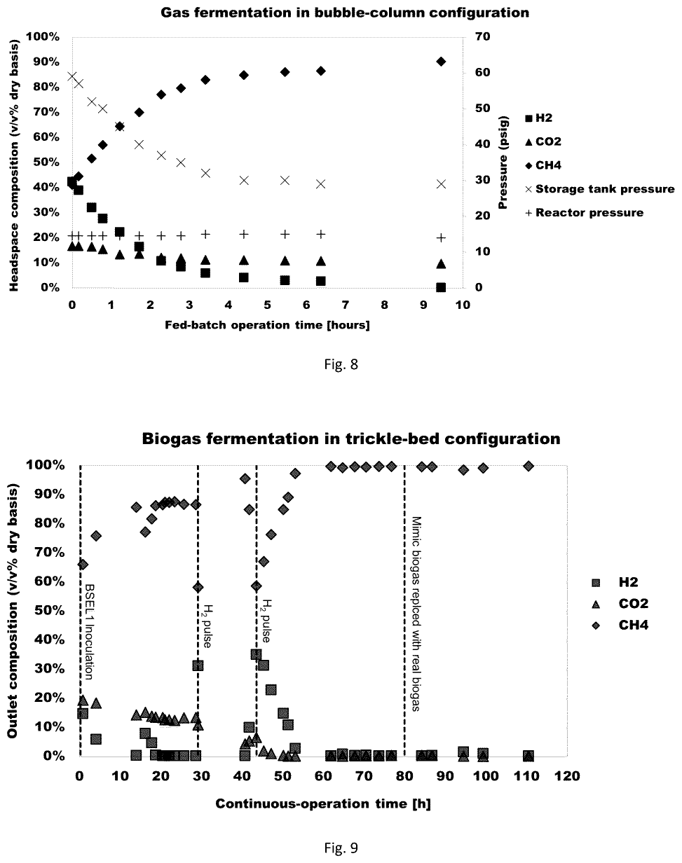

FIG. 10. Schematic depiction of an exemplary bioreactor system.

FIG. 11. Flow diagram of Methanothermobacter wolfeii BSEL1 was isolated from a thermophilic anaerobic digester via enrichment cultivation.

FIGS. 12A and B. Temperature (A) and pH (B) response curves for M. wolfeii BSEL1.

### DETAILED DESCRIPTION

The present disclosure thus describes the isolation and genetic modification of a new variant of Methanothermobacter wolfeii, M. wolfeii BSEL1, an archaeon that utilizes CO₂ as an electron acceptor to produce methane (CH₄) and water (H₂O). The new variant is advantageously capable of minimal nutrient utilization and yet high CH₄ production when cultivated anaerobically in a minimal media such as mineral media, thereby providing a cost-effective methanogen for CO₂ capture.

Initially, a wild type variant of M. wolfeii was enriched from an anaerobic digester using anaerobic techniques. The original wild type was then genetically modified to produce a mutant capable of growing without additional organic nutritional supplements prior to full isolation. In order to do so, two biological mechanisms were exploited: 1) adaptive laboratory evolution and 2) horizontal gene transfer. Adaptive laboratory evolution selection involves the identification of useful random mutations that take place when cultivating a microorganism repeatedly under the desired conditions, e.g. while diluting the original culture. The mutant recovered after sequential dilutions has higher fitness toward growth in mineral media without supplements. Horizontal gene transfer from other microbes that were present in the microbiome while in culture may also have occurred during the adaptive laboratory evolution procedure. Horizontal gene transfer likely happened prior to full isolation of the mutant strain BSEL1. The methanogen mutant BSEL1 displayed enhanced capabilities to grow at a rapid rate in mineral medium with no organic nutrient addition, as well as in no cost municipal wastewater from a full-scale wastewater treatment facility. BSEL1 grew without organic supplements and surprisingly showed the same growth rate with and without organic supplements. Furthermore, the BSEL1 mutant displayed a high conversion rate of H₂ and CO₂ into CH₄ during different cultivation methods including batch, fed-batch and continuous, as well as several reactor configurations including Balch tubes, bubble-column reactor, and trickling bed reactor.

In exemplary aspects, the microorganisms of the disclosure are autotrophic. As used herein, the term “autotrophic” refers to a microorganism capable of using carbon dioxide, formic acid, and/or carbon monoxide as carbon source, and a source of reducing power to provide all carbon and energy necessary for growth and maintenance of the cell (e.g., microorganism). Suitable sources of reducing power may include but are not limited to hydrogen, formate, acetate, and carbon monoxide or electrodes (where they used the electric current) or a combination thereof. In exemplary aspects, the autotrophic microorganisms of the disclosure obtain reducing power from hydrogen.

In exemplary embodiments, the isolated autotrophic Methanothermobacter microorganism of the disclosure is (a) a microorganism of Methanothermobacter wolfeii strain BSEL, deposited on 16 Aug. 2024, with the American Type Culture Collection (ATCC®) under ATCC® Patent Deposit Designation No. PTA-127814, (b) a variant of this strain, or (c) a progeny of this strain, wherein the variant or progeny retains, at least in part, the CO₂ conversion phenotypic characteristics of the parent strain M. wolfeii BSEL1.

M. wolfeii BSEL1 is referred to herein as “BSEL1” or “BSEL”. The 16S rRNA gene sequence and the whole genome of Methanothermobacter wolfeii BSEL1 have been submitted to NCBI GenBank database under the accession numbers OR552920 for the 16S rRNA gene sequence and JAXUHJ000000000 for the whole genome.

The disclosure also provides a substantially pure culture or monoculture comprising any of the microorganisms of the disclosure and/or is a substantially pure culture. As used herein the term “monoculture” refers to a population of microorganisms derived or descended from a single species (which may encompass multiple strains) or a single strain of microorganism. The monoculture in some aspects is “pure,” i.e., nearly homogeneous, except for (a) naturally-occurring mutations that may occur in progeny and (b) natural contamination by non-methanogenic microorganisms resulting from exposure to non-sterile conditions. Organisms in monocultures can be grown, selected, adapted, manipulated, modified, mutated, or transformed, e.g. by selection or adaptation under specific conditions, irradiation, or recombinant DNA techniques, without losing their monoculture nature.

As used herein, a “substantially pure culture” refers to a culture that substantially lacks microorganisms other than the desired species or strain(s) of microorganism. In other words, a substantially pure culture of a strain of microorganism is substantially free of other contaminants, which can include microbial contaminants (e.g., organisms of different species or strain). In some embodiments, the substantially-pure culture is a culture in which greater than or about 70%, greater than or about 75%, greater than or about 80%, greater than or about 85%, greater than or about 90%, greater than or about 91%, greater than or about 92%, greater than or about 93%, greater than or about 94%, greater than or about 95%, greater than or about 96%, greater than or about 97%, greater than or about 98%, greater than or about 99% of the total population of the microorganisms of the culture is a single, species or strain of methanogenic microorganism. By way of example, in some embodiments, the substantially pure culture is a culture in which greater than 70%, 75%, 80%, 85%, 90%, 91%, 92%, 93%, 94%, 95%, 96%, 97%, 98%, 99% or more of the total population of microorganisms of the culture is a single methanogenic microorganism species, as disclosed herein.

In any of the exemplary embodiments described herein, the microorganism may be isolated. As used herein, the term “isolated” means having been removed from its natural environment, not naturally occurring, and/or substantially purified from contaminants that are naturally associated with the microorganism.

In other embodiments, the culture comprises a plurality of (e.g., a mixture or combination of two or more) different species of methanogenic microorganisms, one of which is BSEL1. In some aspects, the culture comprises two, three, four, five, six, seven, eight, nine, ten, or more different species of methanogenic microorganisms, one of which is BSEL1. In some aspects, the culture comprises a plurality of different species of methanogenic microorganisms, one of which is BSEL1, but the culture is substantially free of any non-methanogenic microorganism.

In yet other embodiments, the culture comprises a plurality of microorganisms of different species, in which at least one is a methanogenic microorganism of the present disclosure. In some aspects of this embodiment, the culture comprises BSEL1 and further comprises at least one selected non-methanogenic microorganism. In some aspects, the culture comprises two or more different species of methanogens, of which one is BSEL1, and, optionally comprises at least one selected non-methanogenic microorganism.

In exemplary aspects, the substantially pure culture or monoculture comprises (a) BSEL1; (b) a variant of BSEL1, or (c) a progeny of BSEL1, wherein the variant or progeny retains, at least in part, the CO₂ conversion phenotypic characteristics of the parent strain BSEL1. Also encompassed are the organism in various states such as dormancy, in which the disclosed microorganisms are not producing methane; an “operating” or “active state” in which the disclosed microorganisms are producing methane; active or logarithmic growth phase in which the methanogenic microorganisms are actively multiplying at a rapid rate; stationary phase in which the disclosed microorganisms (after the logarithmic or active growth phase), the rate of cell division and the rate of cell death are in equilibrium or near equilibrium, and thus a relatively constant concentration of microorganisms is maintained in the reactor. etc. All such stages are encompassed.

As used herein, the term “progeny” refers to any microorganism resulting from the reproduction or multiplication of M. wolfeii BSEL1, i.e., any descendant of BSEL1. In exemplary embodiments, the progeny are genetically identical to BSEL1, and, as such, the progeny may be considered as a “clone” of BSEL1. In alternative exemplary embodiments, the progeny are substantially genetically identical to BSEL1, such that the sequence of the genome of the progeny is different from the genome sequence of the microorganism BSEL1 (e.g. exhibiting at least about 95% identity, such as about 95, 96, 97, 98 or 99% identity), but the phenotype of the progeny is substantially the same as the phenotype of BSEL1. In exemplary embodiments, the progeny result from culturing the BSEL1 under the conditions set forth herein for the parent BSEL1. In other words, the variant retains the CO₂ conversion phenotypic characteristics of the microorganism of BSEL1, as described herein.

Other forms of BSEL1 include those intended to preserve the archaea for lengthy periods of time, for transportation and shipping, etc. These forms include but are not limited to desiccated and/or freeze-dried forms from which most (at least 75, 80, 85, 90, 95% or more) of the water has been removed; various concentrates of the microorganism; etc. Such forms encompassed the archaea e.g. in the form of granules, pellets, powders, etc.

### Phenotypic Characteristics

The phenotypic characteristics of interest that are present in BSEL1 and variants and progeny thereof include but are not limited to:

i) The ability to grow and produce CH₄ from CO₂ and H₂ in media that is devoid of a dedicated supply of organic macro- and micronutrients, e.g. BSEL1 grows at maximum rate in minimal media, mineral media, wastewater, wastewater with added vitamins, tap water with added vitamins, etc. Under these minimal conditions, the microorganism produces CH₄ at a rate of at least about 66 mL standard temperature and pressure (0° C. and 1 atm) per hour (STP h⁻¹) and up to 434 mL STP h⁻¹ (e.g. about 5.0, 6.0, 7.0, 8.0, 9.0, 10.0, 11.0 or 12.0 mL STP h⁻¹) in systems with less than 4 L of reaction volume without optimized gas-liquid mass transfer. In systems with optimized gas-liquid mass transport, BSEL1 supports a high gas rate and produces CH₄ at a rate of at least 435 mL STP h⁻¹ and up to 969 mL STP h⁻¹.

ii) M. wolfeii BSEL1 grew at pHs between 6.5 and 8.6, with a maximum growth rate of 0.27±0.03 h⁻¹ at pH 8.3.

iii) The growth temperature range of the BSEL1 strain was between 45° C. and 75° C., with optimum growth at 64° C.

iv) BSEL1 growth is non-dependent on tungsten, in contrast to other M. wolfeii strains having metalloenzymes that depend on this metal as a cofactor.

v) Unlike other methanogenic archaea, M. wolfeii BSEL1 stains Gram negative.

vi) The F subunit (fwdF) of M. wolfeii BSEL1 formylmethanofuran dehydrogenase has the amino acids serine, aspartic acid and isoleucine at positions 17, 69, and 228, respectively, and the D subunit (fwdD) has valine at position 46.

In exemplary embodiments, the variant exhibits or comprises certain characteristics or features, which, optionally, may be specific to a given growth phase (active growth phase, stationary growth phase, nearly stationary growth phase) or state (e.g., dormant state, operating state). For example, in some embodiments, the variant microorganism has been modified to survive and/or grow in a desired culture condition which is different from a prior culture condition in which BSEL1 survived and/or grew. The desired culture conditions may differ from the prior environment in temperature, pH, pressure, cell density, volume, humidity, salt content, conductivity, carbon content, nitrogen content, vitamin-content, amino acid content, mineral-content, or a combination thereof. In some embodiments, the methanogenic microorganism, before adaptation in culture or genetic modification, is one that is not a halophile but, through adaptation in culture or genetic modification, has become a halophile. As used herein, “halophile” or “halophilic” refers to a microorganism that survives and grows in a medium comprising a salt concentration higher than 100 g/L.

### Culture Medium

A culture comprising the BSEL1 methanogenic microorganisms, may be maintained in or on a culture medium. In some embodiments, the culture medium is a solution or suspension (e.g., an aqueous solution). In other embodiments, the culture medium is a solid or semisolid. In yet other embodiments, the culture medium comprises or is a gel, a gelatin, or a paste.

In some embodiments, the culture medium is one that encourages the active growth phase of the methanogenic microorganisms. In exemplary aspects, the culture medium comprises materials, e.g., nutrients, in non-limiting amounts that support the various phases or states of BSEL1, such as a relatively high exponential growth phase of BSEL1. The materials and amounts of each material of the culture medium that supports the active phase of the methanogenic microorganisms typically vary depending on the species or strain of a microorganism. However, it is within the skill of the ordinary artisan to determine the contents of culture medium suitable for supporting the active phase of the microorganisms of the culture. In some embodiments, a culture medium supports the exponential growth. Exemplary components of media for BSEL1 include but are not limited to: Inorganic Materials: Inorganic Elements, Minerals, and Salts

In some embodiments, the medium for culturing archaea comprises one or more nutrients that are inorganic elements, or salts thereof. Common inorganic elements include but are not limited to sodium, potassium, magnesium, calcium, iron, chloride, sulfur sources such as hydrogen sulfide or elemental sulfur, phosphorus sources such as phosphate and nitrogen sources such as ammonium, nitrogen gas or nitrate. Exemplary sources include NaCl, NaHCO₃, KCl, MgCl₂, MgSO₄, CaCl₂, FeSO₄, Na₂HPO₄, NaH₂PO₄, H₂S, Na₂S, NH₄OH, N₂, and NaNO₃. In some embodiments, the culture medium further comprises one or more trace elements selected from the group consisting of ions of barium, bromine, boron, cobalt, iodine, manganese, chromium, copper, nickel, selenium, vanadium, titanium, germanium, molybdenum, silicon, iron, fluorine, silver, rubidium, tin, zirconium, cadmium, zinc, tungsten and aluminum. These ions may be provided, for example, in trace element salts,

In some embodiments, the medium comprises one or more minerals selected from the group consisting of nickel, cobalt, sodium, magnesium, iron, copper, manganese, zinc, boron, phosphorus, sulfur, nitrogen, selenium, optionally tungsten, aluminum and potassium including any suitable non-toxic salts thereof. Thus, in some embodiments, the minerals in the medium are provided as mineral salts. Any suitable salts or hydrates may be used to make the medium. In some embodiments, L-cysteine may be added as a reduction-oxidation (redox) buffer to support early phases of growth of a low-density culture.

In some embodiments, the media comprises a nitrogen source, e.g., ammonium hydroxide or ammonium chloride in an amount of about 1 mM to about 10 mM, e.g. 2 mM, 3 mM, 4 mM, 5 mM, 6 mM, 7 mM, 8 mM, 9 mM, or any combination of the foregoing range endpoints.

In some embodiments, the microorganism is adapted to prefer high salt conditions, e.g. about 1.5 M to about 5.5 M NaCl, or about 3 M to about 4 M NaCl. In some embodiments, the microorganism is adapted to growth in higher salt conditions than their normal environment.

Conditions for culturing the microorganisms disclosed herein may vary, depending, e.g. on the source of CO₂, the location of the culture, etc. For example, for laboratory and/or industrial CH₄ production, the culture medium is generally and advantageously, a BA medium comprising the following components expressed as [mg/L]: NH₄Cl [1000]; NaCl [100]; NaHCO₃[2600], MgCl₂*6H₂O [100]; CaCl₂)*2H₂O [50]; K₂HPO₄*3H₂O [400]; H₃BO₃ [0.1]; ZnCl₂ [0.1]; CuCl₂*2H₂O [0.04]; MnCl₂*2H₂O [0.04]; (NH₄)₂MoO₄*4H₂O [0.1]; AlCl₃*6H₂O [0.1]; CoCl₂*6H₂O [0.1]; NiCl₂*6H₂O [0.1]; ethylenediaminetetraacetic acid Na-salt (EDTA) [0.5]; Na₂SeO₃*5H₂O [0.1]; FeCl₂*4H₂O [2]; biotin [0.02]; folic acid [0.02]; pyridoxine hydrochloride [0.1]; riboflavin [0.05]; thiamine hydrochloride [0.05]; cyanocobalamin [0.001]; nicotinic acid [0.05]; p-aminobenzoic acid [0.05]; lipoic acid [0.05]; DL-pantothenic acid [0.05]; and resazurin sodium salt [0.5].

Those of skill in the art will recognize that the precise type of component and the amount of each can vary and are best determined by small-scale testing. For example, other possible components include but are not limited to one or more of: CaSO₄; H₃BO₃; Co(NO₃)₂; and CuSO₄, and various salts and hydrates thereof. The formula may be adjusted, and/or standardized to suit desired performance parameters.

In some exemplary aspects, BSEL1 is cultured in a minimal media (e.g. for example, tap water) with added vitamins. Vitamins which are added under these growth conditions may include but are not limited to: ascorbic acid, biotin, choline chloride; pantothenate, folic acid, i-inositol, menadione, niacinamide, nicotinic acid, para-aminobenzoic acid (PABA), pyridoxal, pyridoxine, riboflavin, thiamine-HCl, vitamin A acetate, vitamin B12 and vitamin D2. In some embodiments, the medium is supplemented with a vitamin that is essential to survival of the methanogenic microorganism, but other vitamins are substantially absent. In some aspects, the vitamins include but are not limited to riboflavin; thiamine; cyanocobalamin; one or more B-complex vitamins such as nicotinic acid (vitamin B3), and the like.

Typically, the culture medium requires a source of H₂. Hydrogen gas may be produced from a variety of sources. In one embodiment, inexpensive electric power can be used to produce hydrogen from water via electrolysis. For example, an integrated electrolysis/methane fermentation system can be viewed as converting an intermittent energy source (e.g. inexpensive off-peak electricity from power plants) to a stable chemical energy store, using hydrogen as an intermediate and methane as the final energy carrier. Other sources include the thermal breakdown of water, water radiolysis, steam reforming of natural gas, biogenic H₂ produced by acidogenic bacteria, and the like.

Further, in some aspects, the culture medium is or comprises what would otherwise be waste CO₂, i.e., a waste stream, such as waste water, partially treated waste water, sewage, sewage sludge, sewage sludge digester gas, contaminated liquids, atmospheric CO₂, CO₂ extracted from a source where it is unwanted and stored underground, combustion flue gases, etc.

The pH of the culture medium generally ranges from about 7.0 to about 9.0, such as about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8 or 9.0. The range is generally from about pH 8.0 to about 8.6, such as about 8.3.

The temperature of cultivation is generally from about 55 to about 75, such as about 55, 56, 57, 58, 59, 60, 61, 62, 63, 64, 65, 66, 67, 68, 69, 70, 71, 72, 73, 74, or 75. The temperature range is generally from about 60 to about 70, such as about 64.

Other possible media components and culture conditions for methanogens in general are described in detail, for example, in published United States patent application US20220170050, the complete contents of which is hereby incorporated by reference in entirety.

In some embodiments, components required for the growth of non-methanogenic organisms are substantially absent from the media. Such components include, but are not limited to, one or more organic carbon sources, and/or one or more organic nitrogen sources, and/or one or more vitamins and/or tungsten. In some embodiments, formate, acetate, ethanol, methanol, methylamine, and any other metabolically available organic materials are substantially absent from the media.

In some aspects, tungsten is absent from the media.

In some embodiments, the cultures are maintained in a culture vessel within a pressure range from about 0.5 atmospheres to about 500 atmospheres. The culture can be maintained with a source of intermittent agitation, shaking, stirring, and the like. Also in exemplary embodiments, the methane gas removed from the culture suitably comprises less than about 450 ppm hydrogen sulfide, or alternatively less than about 400 ppm, 300 ppm, 200 ppm, 150 ppm, 100 ppm, 50 ppm or 20 ppm of hydrogen sulfide. Total gas delivery rates (CO₂) in the range of 0.2 to 4 volume of gas (STP) per volume of culture per minute are suitable, since they both maintain and exploit high volumetric concentrations of active methane generation capacity.

### Exemplary Systems

The disclosure furthermore provides systems and/or apparatuses and methods for converting carbon dioxide into methane. The systems include, and the methods employ, a supply of carbon dioxide, a source of reducing power, a microorganism in accordance with the disclosure or a variant or progeny thereof, as described herein, within a biological reactor. A biological reactor, also known as a fermentor vessel, bioreactor, or simply reactor, as set forth herein may be any suitable vessel in which methanogenesis can take place. Suitable biological reactors should be sized relative to the volume of the CO₂ source. Typical streams of 2,200,000 lb CO₂/day from a 100,000,000 gal/yr ethanol plant would require a CO₂ recovery/methane production fermentor of about 750,000 gal total capacity. Fermentor vessels similar to the 750,000 gal individual fermentor units installed in such an ethanol plant would be suitable.

In exemplary aspects, the source of reducing power (in whole or in part) is hydrogen, e.g., hydrogen (H₂) gas. Accordingly, in exemplary aspects, the system (e.g. a bioreactor) of the present disclosure includes a supply of carbon dioxide, a supply of hydrogen gas, and a microorganism in accordance with the disclosure or a variant or progeny thereof, as described herein. Examples of suitable bioreactors include but are not limited to: stratified bioreactors, cascaded bioreactors, electro-biological apparatuses, e.g. Systems and apparatuses of this category are further described in International Patent Application No. PCT/US2010/040944, filed Jul. 2, 2010, and published United States patent application US20210115477A1, the complete contents of each of which is incorporated herein by reference in its entirety. Generally, the methanogenic microorganisms disclosed herein are cultured, for example, in shake or stirred tank bioreactors, hollow fiber bioreactors, or fluidized bed bioreactors, and operated in a batch, fed batch, continuous, semi-continuous, or perfusion mode.

In an exemplary method, methane is produced from electric power in a two-step process in a bioreactor. The first step uses electric power to make hydrogen gas from water in a standard water electrolysis system. In a second step, the hydrogen gas is then pumped into a methanogenic reaction chamber, such as a biological reactor as is described in greater detail in U.S. Publ. No. 2009/0130734 which is incorporated in its entirety herein by reference. The reaction chamber comprises a culture of the microorganisms described herein, e.g. within a container that forms part of the bioreactor.

A bioreactor, also known as a digestor or fermenter vessel, as set forth in this disclosure is any suitable vessel in which methanogenesis can take place. Suitable bioreactors should be sized relative to the volume of the carbon dioxide source balanced with its specific need for hydrogen. Typical streams of 2,200,000 lb carbon dioxide/day from a 100,000,000 gal/yr ethanol plant would require a carbon dioxide recovery/methane production bioreactor of about 750,000 gal total capacity. Vessels similar to the 750,000 gal individual fermentor units typically installed in such an ethanol plant may thus be suitable.

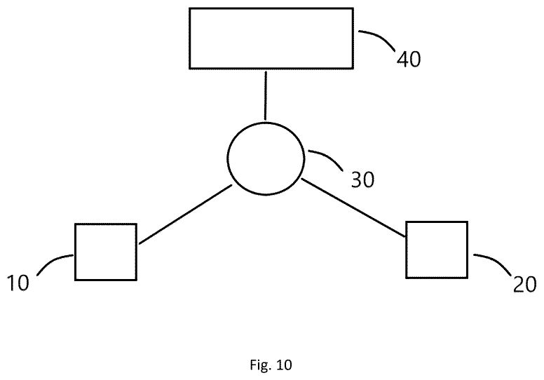

An exemplary bioreactor system is depicted schematically in FIG. 10. The bioreactor system comprises H₂ source 10, CO₂ source 20 and reaction chamber 30. Methane gas produced in reaction chamber 30 is collected in CH₄ collection vessel 40.

In some aspects, a manufacturing plant, e.g. a fuel methane plant, uses the microorganisms described herein. The input to the plant includes e.g. an industrial CO₂ source from combustion or biological produced CO₂ from conversion of organics. An electrolyzer produces hydrogen, which is also collected and stored before use in the plant. The hydrogen and carbon dioxide from their respective storage tanks are optionally fed through an oxygen scrubber for removal of oxygen from the carbon dioxide. After passing through the oxygen scrubber, the hydrogen and carbon dioxide are fed into a digestor/fermentor/bioreactor system for conversion of carbon dioxide and hydrogen to methane. The methane gas vented from the system optionally passes through a sulfur scrubber for recovering sulfur from the product methane stream. The methane gas can then be stored in a methane storage tank or injected into the natural gas grid.

Other exemplary systems are described, for example, in published United States patent application US20220170050, the complete contents of which is hereby incorporated by reference in entirety.

### Uses

Gas fermentation with M. wolfeii BSEL1 may be used to convert any or all of various CO₂ “waste” streams/sources to methane, including but not limited to: industrial sources such as syngas, combustion flue gases, steelmaking slag, municipal solid waste incinerator ashes, combustion fly ash, black liquor, paper mill waste, mining waste, cement waste, construction and demolition waste, waste from the organic industry, flue gas desulfurization gypsum waste, CO₂ generated by manufacturing, mining, agriculture, and energy production, production of ethanol, electricity and heat production, transport, combined cycle coal fired energy plants; during space missions such as its use as a biocatalyst to transform CO₂ from e.g., the Martian atmosphere or from the exhaust breath of astronauts (the resulting CH₄ could be liquified and used as rocket fuel or as an alternative source for electricity generation in microturbines); and the like. M. wolfeii BSEL1 may be used in conjunction with or instead of any carbon capture method, e.g. any point source capture technology or any general capture technology, examples of which include but are not limited to: geologic (underground) storage facilities, pipelines, saline formations, etc.

Additional examples of processes that that produce CO₂ that can be captured by the present systems and methods include biomass fermentation to produce liquid fuels and coal and biomass gasification processes. Gasification is a process that converts carbonaceous materials, such as coal, petroleum, petroleum coke or biomass (living or dead biological material), into carbon monoxide, hydrogen and carbon dioxide.

### Example

Phenotypic and Genomic Characterization of Methanothermobacter wolfeii Strain BSEL1, a CO₂-Capturing Archaeon with Minimal Nutrient Requirements

This example describes the phenotypic and genomic characterization of a new Methanothermobacter wolfeii variant with evolutionary adaptations that allow the strain to thrive in an environment with H₂ as the sole electron donor and CO₂ as the only carbon source. Isolation began with serial dilution of anaerobic digestate in minimal medium until a culture capable of growing without any organic carbon addition, even at high dilutions of 10⁻⁸, was developed. Isolation was performed using modified Hungate techniques (Wolfe RS. 2011. Techniques for cultivating methanogens. Methods Enzymol 494:1-22). The isolated archaeal strain was capable of growing at maximum rates in minimum media with CO₂ as the only carbon source and low levels of NH₄Cl as sole nitrogen source. Prolonged cultivation of the new wolfeii isolate using minimum additions of minerals and vitamins in the absence of yeast extract showed a consistently high growth rate, demonstrating that the new isolate was stable under these conditions. Optimum temperature and pH for growth of the new isolate were determined. In addition, the new M. wolfeii BSEL1 isolate was grown in a continuous gaseous bioreactor to demonstrate the potential of this strain for CO₂ conversion with H₂ into CH₄. The whole genome of the new variant was de novo assembled and annotated. A comparative analysis of orthologous genes in Methanothermobacter wolfeii BSEL1 and the type strain DSM 2970 displayed differences in the abundance of relevant genes associated to energy metabolism, amino acids synthesis, post-translational modifications, and nitrogen fixation.

### Adaptation, Isolation, and Identification of the Methanogen

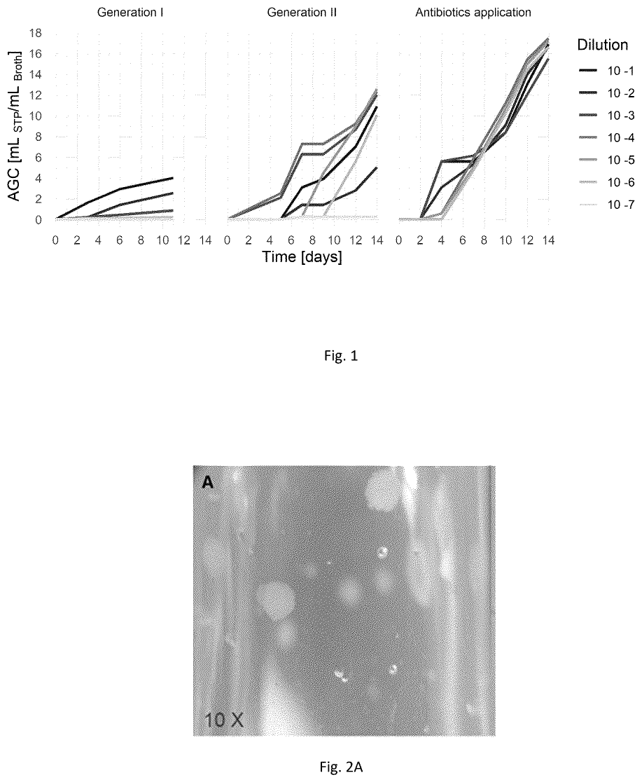

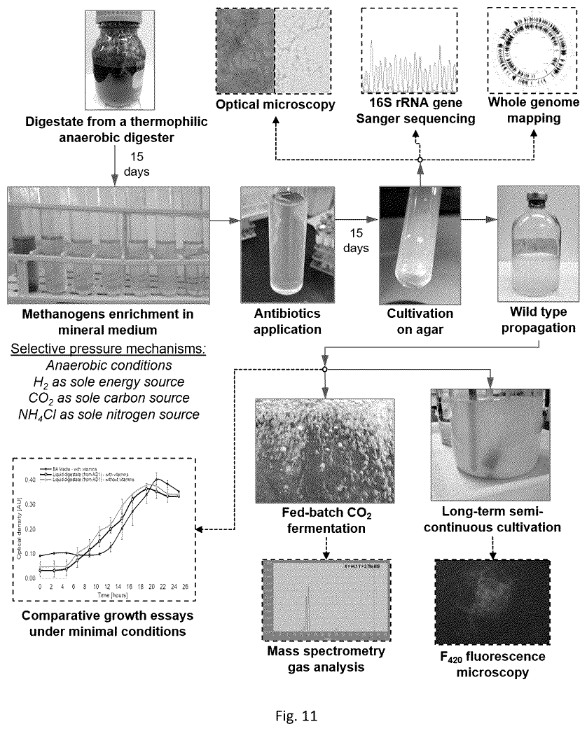

Methanothermobacter wolfeii BSEL1 was isolated from a thermophilic anaerobic digester via enrichment cultivation (See FIG. 11). The isolate was obtained after four serial dilutions without organic supplementation to the medium. Rubber-stoppered Balch tubes were used during the isolation procedure. Basic anaerobic medium (BA medium) was used for isolation by applying modified anaerobic techniques (Wolfe RS. 2011. Methods Enzymol 494:1-22). Before incubation, the headspace of the tubes was replaced and pressurized with H₂ and CO₂ (134.31 kPa absolute pressure). The Balch tubes were incubated at thermophilic conditions (55° C.). The pressure in the tubes decreased over time due to biological gases consumption; this pressure drop was monitored throughout the adaptation and isolation procedure. The pressure drop data were further used to calculate the volumetric gas consumption associated with hydrogenotrophic metabolism (equation 1) (see Materials and Methods section). FIG. 1 shows the resulting accumulated gas consumption (AGC) throughout various generations of the enrichment culture. In generation I, the gas consumption rate decreased in proportion with the dilution factor. Maximum rates of 5.3, 4.0, and 0.9 mL STP day⁻¹ (Standard temperature and pressure conditions (0 deg C. and 1 atm)) were measured for the dilution factors 10⁻¹, 10⁻², and 10⁻³, respectively. Gas consumption was not detected at 10⁻⁴ and higher dilution ratios in generation I. Later, in generation II, higher gas consumption rates were observed in more diluted samples. For instance, the samples within dilution factors of 10⁻⁵ and 10⁻⁶ had gas consumption rates of 10.5 and 8.7 mL STP day⁻¹, respectively. The increase in gas consumption rates between generation I and generation II indicated progress in the adaptation of the methanogenic population(s) to grow under low levels of nutrients. Antibiotics were added to the last two generations of the enriched culture to inhibit bacterial growth while allowing the development of hydrogenotrophic archaea. As shown in FIG. 1, the culture supplemented with antibiotics conserved similar gas consumption rates as that of generation II. In this last generation, gas consumption started relatively early (<4 days) for all the dilutions factors, indicating further enrichment of hydrogenotrophic archaea after the addition of antibiotics.

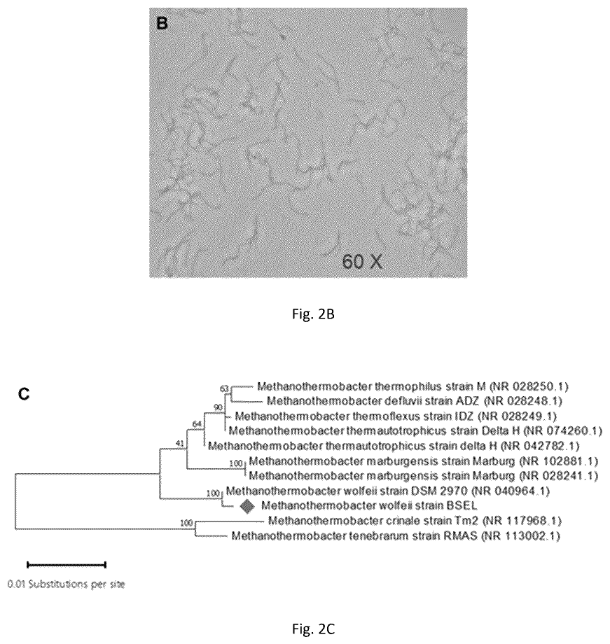

An aliquot from the last generation of the enriched culture was serially diluted and transferred to agar BA medium in roll tubes. The roll tubes were incubated in a pressurized headspace of H₂ and CO₂ (134.31 kPa absolute pressure) at 55° C. for 15 days. At the end of this period, isolated tannish-white colonies growing on the surface and deep inside the agar were observed (see FIG. 2A). The colonies had a circular shape (1.0-2.5 mm diameter), undulated edges, raised elevations, and smooth textures; this description resembles the characteristics of methanogens reported elsewhere. The new isolate manifested a slender rod-shape morphology (FIG. 2B) and the stained Gram negative. Methanogenic activity of the colonies was confirmed with the presence of >20% CH₄ in the headspace of the roll tubes. One of the colonies was picked in aseptic conditions and transferred to fresh liquid BA medium for propagation. The wild type was later cryopreserved in BA medium with added 30% glycerol. Cells were harvested from a glycerol stock to later sequence the 16S rRNA gene.

The new isolate showed 99% nucleotide sequence identity with the taxon Methanothermobacter wolfeii. As shown in FIG. 2C, a phylogenetic analysis confirmed that the new isolate belongs to the same taxon at the species level as the type strain M. wolfeii DSM 2970. We proposed the name Methanothermobacter wolfeii BSEL1 for the new isolate, as it was obtained in the Bioproducts Sciences and Engineering Laboratory (Washington, USA).

Optimal Growth Conditions of Methanothermobacter wolfeii Strain BSEL1

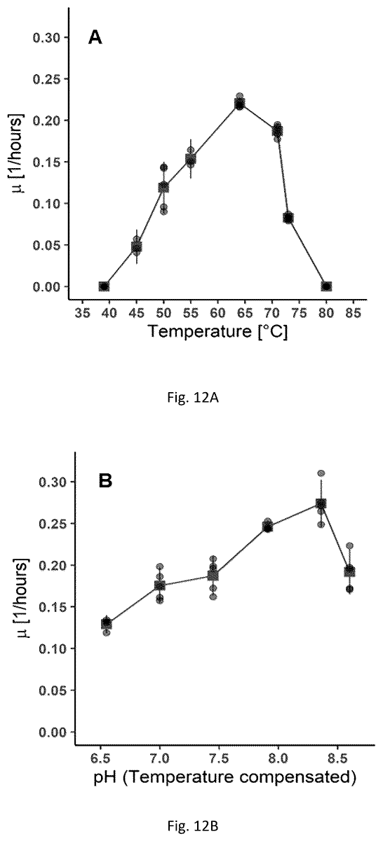

The optimal temperature and pH conditions to cultivate M. wolfeii BSEL1 were determined using optical density (OD590) as the response variable in quantitative growth assays (n=3-5). The specific growth rate (μ) was calculated using the logistic growth model (equation 3) (see Materials and Methods section). The temperature and pH response curves for M. wolfeii BSEL1 are shown in FIGS. 12A and B. The growth temperature range of the BSEL1 strain was between 45° C. and 75° C., with optimum growth at 64° C. M. wolfeii BSEL1 grew at pHs between 6.5 and 8.6, with a maximum growth rate of 0.27±0.03 h⁻¹ at pH 8.3. Table 1 summarizes these and other phenotypic characteristics of M. wolfeii BSEL1 in comparison with other M. wolfeii isolates reported in the literature. Motility, fimbriae or canulae and endospore formation was absent for DSM 2970, OCM36 and BSEL1 and not reported for SIV6.

TABLE 1
Phenotypic characteristics of Methanothermobacter wolfeii and variants

| | | Cell | Optimum | Optimum | Maximum μ |
|---|---|---|---|---|---|
| Isolate | Source | morphology | Temp. (° C.) | pH | reported (h⁻¹) |
| DSM | Mix of river | Slender rods | 65 | 7.0-7.5 | 0.17 |
| 2970* | sediment and | in chains | | | |
| | sewage sludge | | | | |
| | (Germany) | | | | |
| OCM36** | Sewage sludge | Slender rods | 56 | 8.0 | nr |
| | digestate | in crooked | | | |
| | (China) | filaments | | | |
| SIV6*** | Sewage sludge | nr | nr | nr | nr |
| | digestate | | | | |
| | (Germany) | | | | |
| BSEL1 | Manure and | Slender rods | 64 | 8.3 | 0.27 ± 0.03 |
| (This | sewage | in crooked | | | |
| work) | sludge digestate | filaments | | | |
| | (USA) | | | | |

nr = not reported
*Winter et al. 1984. Syst Appl Microbiol 5: 457-466.
**Zhao et al. 1986. Applied and Environmnetal Microbiol 52: 1227-1229.
***Maus et al. 2016. Biotechnol Biofuels 9: 171.

### Effect of Nutrients on the Growth Rate

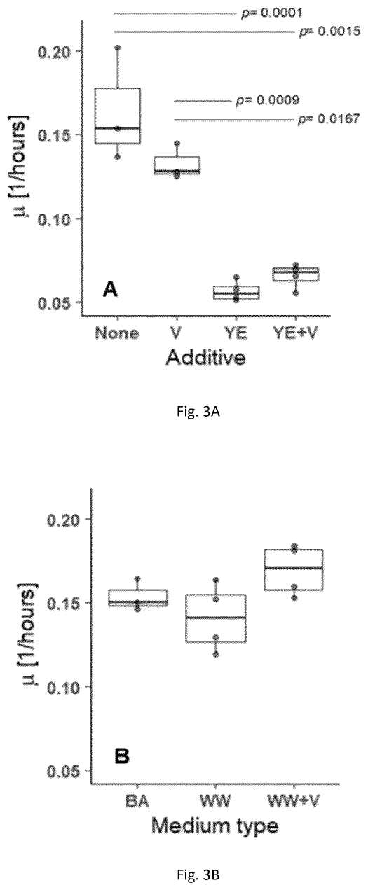

To assess the effect of YE and vitamins on M. wolfeii BSEL1, a set of growth assays were performed in the presence of these additives at 55° C. A set of samples without adding YE and vitamins to the medium was used as a control (see FIG. 3A). This control contained the diluted vitamins from the inoculum (10% dilution factor). The resulting values for each treatment in this experiment were 0.07±0.01, 0.06±0.01, 0.13±0.02, and 0.16±0.05 h⁻¹ for YE and vitamins, only YE, only vitamins, and control, respectively (n=3-4). The addition of YE significantly inhibited the growth of M. wolfeii BSEL1 by approximately 56% (Conover post hoc test P-value<0.0002). No significant difference was found between the treatment adding vitamins and the control (Conover post hoc test P-value>0.05). Digested sewage sludge was sampled from a full-size anaerobic digestion facility. This material was centrifuged simulating the dewatering process that takes place in a municipal wastewater treatment facility. The supernatant (referred to in this example as “wastewater”) was used as medium in another growth experiment. The effect of wastewater on the specific growth rate of M. wolfeii BSEL1 was tested using BA medium as a control (see FIG. 3B). The resulting values were 0.15±0.02, 0.14±0.03, and 0.17±0.03 h⁻¹ for regular BA medium, wastewater, and wastewater+vitamins, respectively (n=3-4). No significant differences were found among these treatments (Kruskal-Wallis P-value>0.05). The growth rate of 0.14 h⁻¹ on wastewater without vitamin supplementation was confirmed by transferring the culture to a new experiment to further dilute the vitamins carried over from the inoculant.

To determine the influence of low concentrations of NH₄Cl as sole nitrogen source on the growth rate of M. wolfeii BSEL1, a growth assay was conducted using various NH₄Cl concentrations ranging from 1.9 to 10.3 mM in BA medium (see FIG. 3C). The inoculum for this experiment was previously cultivated in 18 mM NH₄Cl, and then transferred to the test tubes containing BA medium with the various concentrations of NH₄Cl. The NH₄Cl concentrations shown in FIG. 3C include the contributions of the inoculum to the initial NH₄Cl concentration in the experimental units. This experiment revealed that M. wolfeii BSEL1 grew at a rate of 0.13 h⁻¹ at 55° C. independent from the concentration of NH₄Cl in the medium.

By analyzing the linearized growth curves from the previous experiment (see FIG. 3D), potential relationships between the growth parameters of the BSEL1 strain and the nitrogen concentration were identified. To evaluate these potential relationships, the data for lag-phase duration (tlag) and specific growth rate that was collected at different nitrogen concentrations were fitted to a range of mathematical models to identify the models with the highest correlation between growth parameters and the NH₄Cl content. The two models with the highest correlation were a polynomial mode M. wolfeii BSEL1 grew at a relatively constant growth rate independently of the inorganic nitrogen content. The performance of other mathematical models was also evaluated, including linear, exponential, logarithmic, polynomial of second-order, and power model, while avoiding higher-order models to prevent over-fitting. These other models had lower correlations.

Longevity of Methanothermobacter wolfeii BSEL1 in Semi-Continuous Cultivation

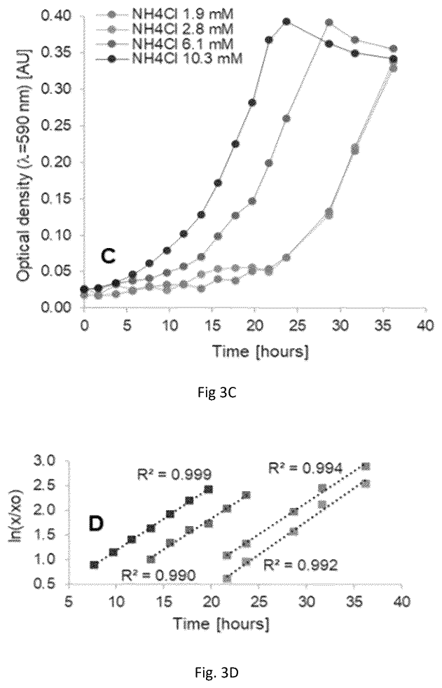

To assess the longevity of M. wolfeii BSEL1 when cultivated under a minimum supply of nutrients, a semi-continuous 2 L bioreactor was inoculated with the BSEL1 isolate and operated at 55° C. with constant agitation (120 rpm) for 8 months. This reactor was fed with a 70% H2:30% CO₂ mixture (134.31 kPa absolute pressure) every 2-3 days. The gas in the headspace of the bioreactor was replaced every feeding cycle with the 70% H2:30% CO₂ mixture to prevent accumulation of CH₄ in the system. Fresh BA medium was added in aseptic conditions once every week with a dilution rate of 1 mL of fresh medium per milliliter of culture broth. At the end of the 8-month period, the culture displayed continuous methanogenic activity, with >50% v CH₄ in the headspace. Besides, fluorescence microscopy revealed the presence of coenzyme F420 in the cells after 8 months of cultivation and only the M. wolfeii BSEL1 morphotype was present (FIG. 4). The presence of acetic acid was assessed by running samples of the culture broth in a high-performance liquid chromatography (HPLC) instrument, which showed absence of acetic acid (detection limit of 5 g/mL). This indicated that the populations of homoacetogens, potential microbial contaminants that compete for H₂ and CO₂, were not active in the bioreactor after 8 months of cultivation.

In addition, it was observed that M. wolfeii BSEL1 maintained viability for more than 48 months of cryopreservation in glycerol. Storage at room temperature in BA medium under a headspace of N₂ was also an effective storage method to preserve the microorganism without signs of viability loss for up to 6 months.

### CO₂-Capture and Methanation Assay in Bubble Column Gas Fermentation

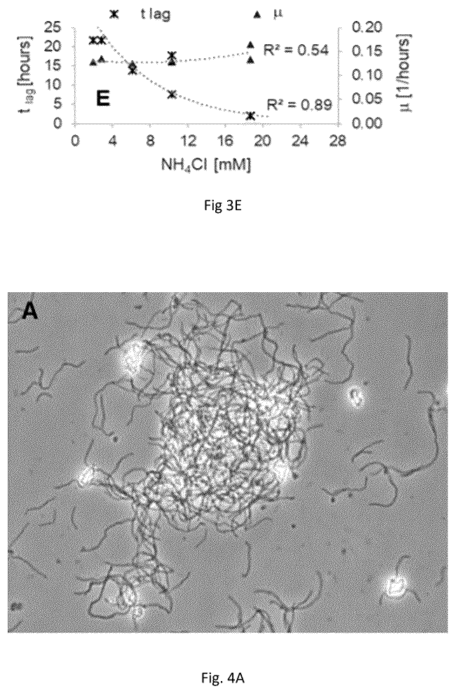

The capability of M. wolfeii BSEL1 as biocatalyst for CO₂ conversion into CH₄ was tested using an 8 L bubble column gas fermentation bioreactor fed with a gas mixture mimicking the biogas from sewage sludge anaerobic digestion (66.5% CH₄ and 33.5% CO₂). This biogas mimic was blended with H₂ to a H₂:CO₂ ratio of 4, in accordance with the stoichiometry of hydrogenotrophic methanogenesis. The gaseous substrate was held in a pressurized tank and sparged at the bottom of the bioreactor. The headspace was kept at a constant pressure of 134.31 kPa, controlled by a pressure switch, without removing gases from the device (except for sampling). Prior to use, the bioreactor was autoclaved at 120° C. for 1 h. Sterile BA medium was added to the bioreactor in anoxic/aseptic conditions before operation. The reactor was operated at 55±0.2° C. and the pH was regulated with a bicarbonate buffer contained in the medium. The composition of the headspace in the bioreactor was analyzed over time using mass spectrometry (see FIG. 5). After 21 h of operation, the gas consumption stopped when the H₂ concentration had dropped to 0.8% H₂. The gas phase at this time contained still 7.2% CO₂ besides 92% CH₄. Subsequently, further H₂ was provided to the reactor to enforce the microbe to consume the remaining CO₂ in the headspace while increasing the final CH₄ titer. After a further 51 h of operation, the final products in the headspace of the reactor were 0.6% H₂, 98.5% CH₄, and 0.9% CO₂.

### Genome Assembly and Genomic Features of the BSEL1 Strain

Whole-genome sequencing of M. wolfeii BSEL1 DNA was performed via next-generation sequencing. 5.8 GB of sequencing data were obtained. FastQC was used for a quality check. Low-quality reads were trimmed using Trimmomatic, conserving 45.3 million reads (forward+reverse) with an average Q-score of 34 (lengths of 30-250 bp). The trimmed reads were de novo assembled using two different tools SPAdes and SKESA. The draft assemblies were examined by comparing their completeness and the copy number of key genes that translate into the main metabolic and stress response functions in methanogens (mcr, acs, frh, por, and trx). The draft assemblies were found to be comparable, both displaying a completeness of 90.6% and the same copy number for all the key genes inspected. Ultimately, the genome assembled with SKESA was selected for downstream processing, as SKESA has been reported to generate 13% less mismatches than SPAdes. The final genome of M. wolfeii BSEL1 consisted of 1.66 kbp. The similarity of conserved regions in the whole-genome sequence of the BSEL1 strain with closer relatives from the Methanothermobacter genus was assessed by calculating the average nucleotide identity (ANI). It was confirmed that the closest phylotype to the BSEL1 strain is M. wolfeii DSM 2970 (ANI of 99.35), followed by Methanothermobacter marbur gensis (83.63) and Methanothermobacter defluvii (83.43). The genome was annotated using the NCBI Prokaryotic Genome Annotation Pipeline (PGAP).

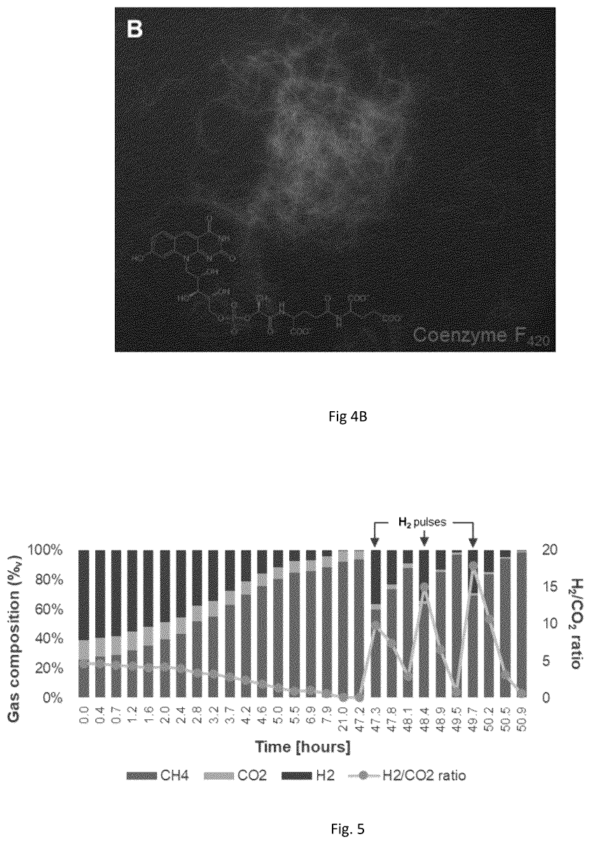

The chromosomal map of the BSEL1 strain is shown in FIG. 6, and a comparison between the BSEL1 strain genome annotation against the type strain is presented in Table 2. A BLAST comparison between the annotated genome and the type strain revealed that 1,452 genes in the BSEL1 strain genome have an identity alignment score of less than 0.6 when compared to the type strain, revealing relevant differences on a genomic level. It is interesting to note that the region between 550 and 680 kbp of the chromosomal map of M. wolfeii BSEL1 displayed a particularly low nucleotide identity compared to the type strain. All the genes with low identity scores located in the mentioned region corresponded to hypothetical proteins.

TABLE 2
Annotation details of M. wolfeii BSEL1 and
M. wolfeii DSM 2970 genomes through NCBI PGAP

| | M. wolfeii | M. wolfeii DSM 2970 |
|---|---|---|
| Features | BSEL1 | (type strain) |
| Size (Mbp) | 1.66 | 1.71 |
| Total genes | 1,842 | 1,890 |
| Protein coding sequences | 1,796 | 1,838 |
| (CDS) | | |
| Hypothetical proteins | 1,411 (79% of | 1,425 (75% of |
| | CDS) | CDS) |
| CDS with unidentified | 917 (50% of | 966 (51% of |
| function (Kyoto | CDS) | CDS) |
| Encyclopedia of Genes and | | |
| Genomes) | | |
| Total non-coding RNA | 41 | 46 |
| genes | | |
| GC content | 48.9% | 48.5% |
| Completeness | 90.6% | 89.6% |

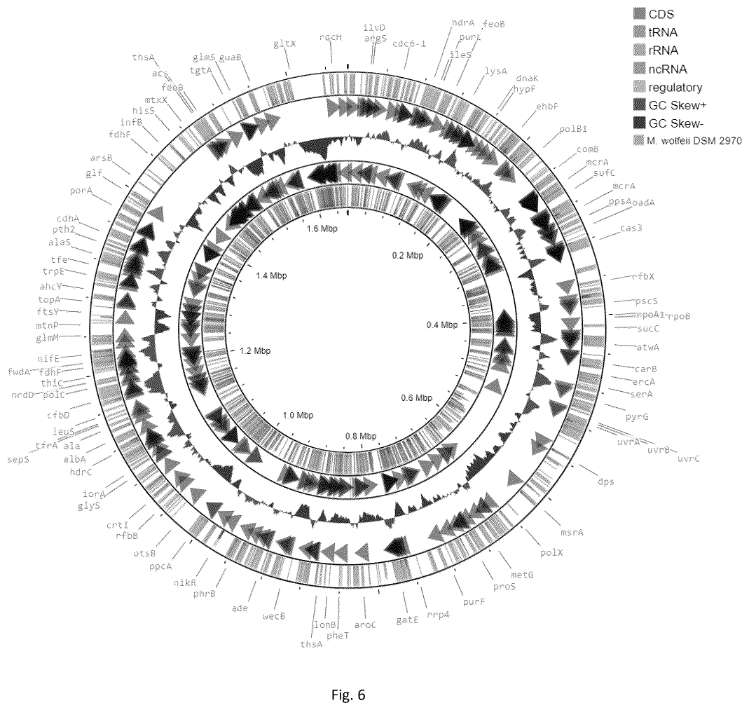

An analysis of orthologous genes was conducted in both the BSEL1 and the type strain by classifying the protein coding genes (coding sequences) of both strains into the functional categories of the Kyoto Encyclopedia of Genes and Genomes (KEGG) as well as the Cluster of Orthologous Genes (COG). This study was tailored to identifying predicted functional features in the BSEL1 strain that may lead to the differences in phenotype with respect to the type stain. The abundances of the various genes/proteins classified in KEGG categories and COG classes are shown in FIG. 7. The COG categories that displayed the highest differential abundance when comparing both strains were energy production and conversion, signal transduction, lipids metabolism, unknown functions, and non-classified proteins. The KEGG categories with the highest differential abundance were glycan biosynthesis and metabolism and energy metabolism. In all the classified KEGG categories, the BSEL1 strain had a higher abundance of features than the type strain. In more detail, the overabundant putative functions and associated enzymes in the BSEL1 strain genome were proteins glycosylation and N-glycan biosyn thesis (dolichyl-diphosphooligosaccharide-protein glycosyltransferase; K07151), lysine biosynthesis (homoserine dehydrogenase; K00003), oxidative phosphorylation (NADH-quinone oxidoreductase subunit F; K00335), nitrogen fixation (nitrogenase iron protein NifH; K02588), and peptidoglycan biosynthesis (UDP-N-acetylmuramoyl-L-alanyl-D-gluta mate-2,6-diaminopimelate ligase; K01928).

### DISCUSSION

Methanothermobacter members are ubiquitous in anaerobic environments. Their genomic signature has been identified in numerous habitats such as lake sediments, hot springs, water-flooded oil fields, and mammalian guts, as well as human-shaped ecosystems such as agricultural soils, landfills, and anaerobic digesters. Hydrogenotrophic methanogens occupy approximately 1% of the microbiome of anaerobic digesters. The enrichment cultivation pipeline shown in FIG. 11 was not only successful to isolate an axenic culture of Methanothermobacter wolfeii from an anaerobic digester, but it also allowed us to isolate a strain with no needs for addition of organic supplements. This adaptation was evidenced on a phenotype level by the increased viability of the methanogen at more diluted samples over the enrichment culture generations (FIG. 1) as well as on a genomic level by the differential abundance of functional genes in the BSEL1 strain (FIG. 7). Inferred adaptation mechanisms responsible for increased fitness of M. wolfeii BSEL1 toward absence of organic supplements are (i) selection of useful random mutations that take place when cultivating a microorganism repeatedly; these mutations ultimately produced a mutant strain with higher fitness toward low concentrations of nutrients; this is also known as adaptive laboratory evolution horizontal gene transfer from other organisms that were present in the microbiome at the moment of culture transfers during the evolution procedure.

The amino acids sequences of the genes with identity scores lower than 0.5 located in the non-conserved 550-680 kbp region of the BSEL1 strain genome were recognized and queried with BLASTp. Some of the putative proteins encoded in the mentioned region included YkvA family protein, ATP-dependent DNA helicase PcrA, DUF169 and DUF2085 domain-containing proteins, carboxypeptidase regulatory-like domain-containing protein, ABC transporter ATP-binding protein, zinc ribbon domain-containing protein, pseudomurein-binding protein, tetratricopeptide repeat protein, RAD55 family ATPase, along with hypothetical proteins with unknown functions. Most of these proteins were discovered in other members of the same genus such as Methanothermobacter sp. THM-1, Methanothermobacter sp. CaT2, and Methanothermobacter thermautotrophicus, as well as metagenomic material from uncultured archaea from various phyla (Euryarchaeota and Thermoproteota) found in thermophilic anaerobic environments, thus supporting the occurrence of horizontal gene transfer evolutionary events from close neighbors cohabiting the anaerobic digester from where the BSEL1 strain was isolated.

Morphologically, the BSEL1 strain is similar to other Methanothermobacter members. All of them lack motility, do not have archaeal appendages (such as fimbriae and canulae), and do not generate endospores (see Table 1). On the other hand, relevant physiological differences were found between M. wolfeii BSEL1 and other isolates from the same species. For instance, the maximum specific growth rate reported for the type strain DSM 2970 was 0.17 h⁻¹ in the presence of YE, while the BSEL1 strain grew at 0.27 h⁻¹ at optimal temperature and pH without the addition of organic carbon and nitrogen to the medium (as shown in FIGS. 12A and B). Another significant difference was that the BSEL1 strain is not dependent on the addition of tungsten, whereas the type strain DSM 2970 possesses metalloenzymes that depend on this metal as cofactor. More about this independence from tungsten in the BSEL1 isolate is further discussed below. Unlike other methanogenic archaea, M. wolfeii BSEL1 stains Gram negative indicating a potential change in the cell wall when the methanogen grows in the absence of organic nutrients. However, Gram stain is specific for evaluating the “thickness” of the peptidoglycan layer in the bacterial cell envelope and it is not recommended for archaea, which lack peptidoglycan. The abundance of pseudomurein in archaeal species can be assessed using HPLC of hydrolyzed cell walls, as suggested by Kandler and Konig.

The lack of nutrients during the isolation procedure likely favored the selection of a mutant adapted to grow on CO₂ as the only carbon source with NH⁴⁺ as the nitrogen source. Interestingly, this implies that M. wolfeii BSEL1 can synthesize the necessary precursors for its biosynthesis processes, including proteinogenic amino acids, solely from inorganic molecules. In contrast, the type strain M. wolfeii DSM 2970 and the model organism M. thermautotrophicus were both isolated using media that contained formate as an alternative carbon source and YE, which provided additional organic carbon and nitrogen, including amino acids. Thus, it is unclear if M. wolfeii DSM 2970 and M. thermautotrophicus could synthesize all the necessary building blocks for cell growth without the addition of organic precursors to the medium. The overabundance of homoserine dehydrogenase in the genome of the BSEL1 strain suggests that the type strain may have a truncated pathway for lysine biosynthesis, thus explaining its dependency on the addition of this amino acid for growth. The minimal medium (BA medium) used to isolate and characterize M. wolfeii BSEL1 in this study had lower concentrations of several minerals when compared with the anaerobic medium originally used to cultivate M. wolfeii DSM 2970 by Winter et al. (see Table 3). These minerals with lower content in BA medium were Na⁺, Mg²⁺, Al³⁺, Ca²⁺, Mn²⁺, Fe²⁺, CO₂⁺, Ni²⁺, Cu²⁺, Zn²⁺, BO³⁻, HCO⁻, and MoO²⁺. This likely indicates that the BSEL1 isolate requires a relatively low content of minerals for growth. Interestingly, the Mg, Ca, Mn, Fe, Cu, and Zn contents in BA medium are equal or lower to the ionic background found in tap water and some bottled waters in the USA. Accordingly, tap water can be sufficient for cultivating the strain when only small amounts of Na, Al, K, Co, Ni, Mo, Cl, B, Se, NH⁺ and vitamins are added. Besides BA medium, all anaerobic media formulations reported in the literature also include resazurin as a redox indicator and cysteine and/or Na₂S as a reducing agent(s). These are unavoidable components to ensure anaerobic conditions.

TABLE 3
Minimal conditions for Methanothermobacter wolfeii cultivation
reported in the literature, in contrast with the present work

| | Organic growth | Inorganic | | | | | |
|---|---|---|---|---|---|---|---|
| | factors and/or | nitrogen | | | | Doubling | Isolate |
| Mineral nutrients | organic nitrogen | source | e⁻ | Temp | Initial | time | designation |
| [mM] | sources | [mM] | donor | (° C.) | pH | (h) | reference |
| NaOH [50], NaHCO₃ [71], NaCl [8.8], | Vitamins + | NH₄⁺ [3.8] | H₂ | 45 | 7.0 | 8 | DSM |
| CaCl₂ [0.49], K₂HPO₄ [1.5], KH₂PO₄ | 0.2% YE + | | | 55 | | 4 | 2970; |
| [1.9], MgSO₄ [0.66], CoCl₂ [0.008], | 0.2% peptone | | | 60 | | 4 | type |
| FeSO₄ [0.013], MnSO₄ [0.033], | | | | 65 | | 4 | strain |
| ZnSO₄ [0.006], CuSO₄ [0.001], | | | | 72 | | 12 | (*) |
| AlKSO₄ [0.0004], Na₂WO₄ [0.0038], | | | | | | | |
| H₃BO₃ [0.0016], Na₂MoO₄[0.0005] | | | | | | | |
| NaHCO₃ [31], NaCl [19], MgCl₂ | Vitamins | NH₄⁺ [18.7] | H₂ | 45 | 6.8 | 14.7 ± 6.1 | BSEL1 |
| [0.5], CaCl₂ [0.3], K₂HPO₄ [1.8], | Vitamins | | | 50 | | 6.1 ± 1.7 | (This |
| CoCl₂ [0.0002], MnCl₂ [0.0003], FeCl₂ | 10% Vitamins | | | 55 | | 4.3 ± 1.3 | work) |
| [0.01], ZnCl₂ [0.0004], AlCl₃ [0.0004], | Vitamins | | | | | 4.5 ± 0.7 | |
| CuCl₂ [0.0002], NiCl₂ [0.0004], | Vitamins + | | | | | 10.6 ± 2.0 | |
| Na₂SeO₃ [0.0004], H₃BO₃ [0.0008], | 0.1% YE | | | | | | |
| (NH₄)₂MoO₄ [0.0002] | 10% Vitamins + | | | | | 12.4 ± 2.0 | |
| | 0.1% YE | | | | | | |
| | Vitamins | | | 64 | | 3.2 ± 0.1 | |
| | Vitamins | | | 71 | | 3.7 ± 0.2 | |
| | Vitamins | | | 73 | | 8.4 ± 0.4 | |
| | Vitamins | | | 64 | 8.3 | 2.5 ± 0.3 | |
| | Vitamins | NH₄⁺ [10.3] | | 55 | 6.8 | 5.3 ± 0.5 | |
| | | NH₄⁺ [6.1] | | | | 5.5 | |
| | | NH₄⁺ [2.8] | | | | 5.2 | |
| | | NH₄⁺ [1.9] | | | | 5.4 | |

| | | |
|---|---|---|
| Wastewater | 7.2 | 5.0 ± 1.2 |
| Wastewater + vitamins | 7.2 | 4.1 ± 0.6 |

^{a}Ranges (±) represent confidence intervals from the mean (a = 0.05, n = 3-5). Composition of vitamins in the medium is reported in Materials and Methods.
*Winter et al. 1984. Syst Appl Microbiol 5: 457-466.

Minerals play a vital role in the metabolism of prokaryotic cells. Among other functions, minerals serve as cofactors for metalloenzymes and as signaling molecules in crucial regulatory pathways of methanogenesis. Methanothermobacter wolfeii was originally described by Winter et al. as a tungsten-dependent methanogen. Here, we show that this characteristic is not shared among all variants of the species, as M. wolfeii BSEL1 grew at maximum rates in BA medium, which lacked tungsten (see Table 3). This metal is responsible for the electron transfer in the active site of ferredoxin oxidoreductases and formyl dehydrogenases. These groups of enzymes have main roles in the Wolfe cycle. For instance, formylmethanofuran dehydrogenase (EC 1.2.7.12) catalyzes the CO₂ fixation step of methanogenesis. Formylmethanofuran dehydrogenase is often described as tungsten-dependent. However, Bevers et al. showed that some formylmethanofuran dehydrogenases in anaerobic archaea use molybdenum, which has the same number of valence electrons as tungsten. Interestingly, the amino acids sequences of all the subunits of formylmethanofuran dehydrogenase encoded in M. wolfeii BSEL1 genome present multiple substitutions when compared with the peptide sequences of the type strain. For instance, the subunit F (fwdF) has the substitutions T→S, N→D, and V→I at the positions 17, 69, and 228 of the peptide sequences, respectively, while the subunit D (fwdD) has the substitution A→V at the position 46. These substitutions may be responsible for the allocation of molybdenum in the formylmethanofuran dehydrogenases as well as the tungsten independence of the BSEL1 strain.

The biosynthesis of amino acids in microorganisms is of conspicuous importance, as it constitutes ca. 20% of the total energy spent during protein synthesis. Methanogens use the Wood-Ljungdahl pathway for CO₂ and H₂ fixation into formate. Later on, formate is transformed into other organic intermediates aided by the central carbohydrate metabolism. Glycolysis, the tricarboxylic acid (TCA) cycle, and the pentose phosphate pathway lead the formation of precursors for the synthesis of amino acids. The only amino acids previously known to be synthesized fully autotrophically, directly from CO₂ and NH⁺ in M. wolfeii DSM 2970, are arginine and proline. Based on the aforementioned, it is likely that all methanogenic species are capable of synthesizing amino acids solely from inorganic primary substrates; however, as shown by Winter et al., their growth rate without the addition of organic carbon sources is significantly reduced. This is, however, not the case of the BSEL1 strain. The addition of YE to the medium for prokaryotes cultivation is a collective practice in microbiology, including practices to cultivate hydrogenotrophic methanogens. Yet, in this work, we found that M. wolfeii BSEL1 was inhibited even by low concentrations of YE (0.1%) (FIG. 3A). YE is a complex additive composed of cell materials extracted from hydrolyzed yeast. YE provides easily fermentable organic carbon and nitrogen compounds including sugars, amino acids, peptides, nucleotides, lipids, and vitamins, as well as certain amounts of minerals. As shown in FIG. 3A, M. wolfeii BSEL1 do not require these additional nutrients and, on the contrary, it appears to be sensitive to them. This observation was unexpected, as previous studies indicated that M. thermautotrophicus was stimulated by the addition of YE (0.05%-0.2%) and M. wolfeii DSM 2970 required YE (0.2%) for fast growth. Toxicity of YE has been reported previously for other archaea, however. The authors associated the inhibitory effect of YE with the chelating effect of organic compounds, which decreases the bioavailability of essential minerals in the medium. The inhibitory effect of YE on M. wolfeii BSEL1 could be associated to similar mechanisms, potentially involving complexation of ions like Fe²⁺ or Cu²⁺ that are necessary for methanogenic metabolism.

The duplication time of M. wolfeii BSEL1 on wastewater with vitamins added (4.1±0.6 h) was equivalent to BA medium (4.5±0.7 h) at the same temperature (see FIG. 3B). This indicates that wastewater could be used as a low-cost medium to cultivate M. wolfeii BSEL1. Wastewater could be withdrawn from conventional centrifugation equipment already existing in wastewater treatment facilities. Wastewater treatment facilities could operate bioreactors with M. wolfeii to convert biogas and H₂ into RNG.

The results presented in FIG. 3E showed that the t lag of M. wolfeii BSEL1 is inversely correlated to the concentration of NH₄Cl. This is likely because the microbe requires more time to accumulate enough NH⁺—N prior to initiating protein synthesis when facing low nitrogen contents, ultimately delaying exponential replication. Nevertheless, t lag does not influence the conversion rates in continuous bioprocesses, as it is not a kinetic constant in the Monod model and its derivations. Therefore, 1.9 mM NH₄Cl is expected to be enough to allow M. wolfeii BSEL1 to maintain a volumetric CO₂ capture rate in a fully conditioned gas fermentation bioreactor in steady-state. On the other hand, the gas-liquid mass transport of H₂ is expected to be the bottleneck for reaction and growth rates during continuous gas fermentation.

During the first 21 h of cultivation of M. wolfeii BSEL1 in the bubble column bioreactor, it was estimated that ca. 0.9 mmol CO₂ L-1 was liberated from the HCO— present in BA medium due to consumption of the bicarbonate buffer. This further leads to a decrease of the H₂:CO₂ ratio, similar to the observations of other authors (see FIG. 5). This is an undesired reaction because CO₂ liberation from HCO⁻ reduces the overall CO conversion efficiency of the bioreactor and increases the H₂ requirements. However, HCO₃⁻ is necessary to provide pH buffer capacity to the medium and avoid a sudden pH increase when CO₂ is consumed by autotrophic metabolism. The use of bicarbonate can be sorted by replacing the buffer system with a phosphate buffer or by taking advantage of the natural buffer capacity of an alternative culture medium such as wastewater. After applying three H₂ pulses, it was observed that the CO₂ concentration in the headspace was reduced to less than 1% v and the final CH₄ titer reached 98.5% v. This product satisfies the standard CH₄ content (>96% v) for injection of RNG to the natural gas pipeline. Further tuning of the process with M. wolfeii BSEL1 achieves the same gas product in continuous operation without the need of additional H₂. The effects of the pollutants found in biogas (e.g., H₂S and/or NH₃) on the growth of M. wolfeii BSEL1 is further investigated.

Gas fermentation with M. wolfeii BSEL1 is extended to other CO₂ industrial streams such as syngas or combustion flue gases. Interestingly, methanogens could also be used as biocatalyst to transform CO₂ into CH₄ during space missions. For instance, CO₂ could be sequestrated from the Martian atmosphere or from the exhaust breath of astronauts and transformed into CH₄. The resulting CH₄ could later be liquified and used as rocket fuel or as an alternative source for electricity generation in microturbines.

### Materials and Methods

### Anaerobic Medium Preparation (BA Medium and Wastewater)

Unless specified differently, BA medium consisted of the following components [mg/L]: NH₄Cl [1,000]; NaCl [100]; NaHCO₃ [2,600], MgCl₂*6H₂O [100]; CaCl₂*2H₂O [50]; K₂HPO₄*3H₂O [400]; H₃BO₃ [0.1]; ZnCl₂ [0.1]; CuCl₂*2H₂O [0.04]; MnCl₂*2H₂O [0.04]; (NH₄)₂MoO₄*4H₂O [0.1]; AlCl₃*6H₂O [0.1]; CoCl₂*6H₂O [0.1]; NiCl₂*6H₂O [0.1]; ethylenediaminetetraacetic acid Na-salt (EDTA) [0.5]; Na₂SeO₃*5H₂O [0.1]; FeCl₂*4H₂O [2]; biotin [0.02]; folic acid [0.02]; pyridoxine hydrochloride [0.1]; riboflavin [0.05]; thiamine hydrochloride [0.05]; cyanocobalamin [0.001]; nicotinic acid [0.05]; p-aminobenzoic acid [0.05]; lipoic acid [0.05]; DL-pantothenic acid [0.05]; and resazurin sodium salt [0.5]. BA medium was made anoxic following modified Hungate techniques: Distilled water was used for medium and stock solutions preparation. Distilled water was pre-reduced by boiling for at least 5 min. The medium was prepared in a flask with a constant flow of pure N₂ delivered through a blunt end needle to minimize air infiltration into the flask during medium preparation. The reducing agent, L-cysteine HCl*H₂O was added to a final concentration of 0.53 g/L. The reduced minimal medium was transferred in aliquots of 4.5 mL to Balch tubes previously flushed with N₂. The tubes were sealed with chlorobutyl rubber stoppers and aluminum crimp seals. The tubes were pressurized to 134.31 kPa with pure N₂ and autoclaved at 121° C. for 20 min. Before use, the medium was given Na₂S (0.06 g/L) and vitamins in the form of filter-sterilized stock solutions. YE added for testing was bacteriological grade and was acquired from Avantor, USA.

The initial inoculant for isolation was taken from a thermophilic anaerobic digester that was operated in our laboratory originally seeded with manure digestate and fed with sewage sludge as raw material with a retention time of 15 days.

To determine the effect of pH on the specific growth rate of M. wolfeii BSEL1, bicarbonate in BA medium was replaced with a bicarbonate-carbonate buffer (0.04 M). The amounts of carbonate and bicarbonate to prepare this buffer were calculated using Henderson-Hasselbalch equation. The pH was adjusted using various partial pressures of CO₂ provided to the Balch tubes while maintaining a constant partial pressure of H₂ (166.44 kPa) among the different treatments. The respective partial pressures of CO₂ were: 71.4 kPa (pH 7.0), 41.4 kPa (pH 7.4), 26.9 kPa (pH 7.8), 19.3 kPa (pH 8.3), and 14.5 kPa (pH 8.5). To adjust the pH to 6.5, the medium was added a lower concentration of bicarbonate (0.02 M) and no carbonate was added. In every case, the pH of the medium was verified in abiotic controls after incubation at 64° C. for 24 h. pH measurements were performed at near 64° C.; the pH measurement at room temperature was discarded as it is not representative of the pH at culture conditions in this case because the temperature changes interfere with the solubility of CO₂. Thus, the pH readings were compensated from the solution temperature to standard temperature (25° C.) using a pH temperature correction calculator. In preliminary tests, it was determined that a simpler approach for adjusting the pH by addition of NaOH (2 M) and HCl (2 M) during the medium preparation was not effective to maintain the pH above 7.3 after incubation with CO₂, likely due to the formation of carbonic acid, when CO₂ dissolves in water (v+H₂O→H₂CO₃→H⁺+HCO⁻), which acidifies the medium. In every case, the tubes were incubated in a horizontal position at 160 rpm (3 cm orbit diameter) to maximize gas-liquid mass transport.

The wastewater used as alternative for the culture medium was obtained by centrifugation at 4,000 g for 10 min of digested sewage sludge sampled from Walla Walla wastewater treatment facility (Washington State, USA). The supernatant (wastewater) was collected, then boiled for 5 minutes, and added with L-cysteine HCl*H₂O to a final concentration of 0.53 g/L. The pre-reduced wastewater was autoclaved at 121° C. for 30 min in Balch tubes that were previously flushed with N₂. Na₂S was added to a final concentration of 0.06 g/L from a filter-sterilized stock solution.

### Pressure Drop and Gas Consumption in Balch Tubes

The pressure in the Balch tubes was measured using a pressure gauge (Grainger, 4FLV8, USA) modified to allocate a Luer-Lok fitting. With this fitting, the pressure gauge was able to tightly hold a sterile hypodermic needle that was later used to penetrate the rubber stopper on the Balch tubes and measure the pressure within. The tubes were allowed to cool down to room temperature for 30 min prior to every pressure measurement. After every pressure measurement, the enrichment cultures were re-pressurized with the 70/30 H₂:CO₂ mixture (134.31 kPa absolute pressure) and returned to the incubator. In every generation, a blank without inoculation (abiotic control) was included to detect and subtract the contribution of gas solubilization to the pressure drop. The gas consumption (Gi) during the enrichment cultivation was calculated from the pressure drop (A P) in the Balch tube using Eq. 1, where ΔP_{Abiotic} is the pressure drop due to gas solubilization (obtained from the abiotic control), V_{H} is the volume of the headspace in the Balch tube, T_{S} is the standard temperature for gases (273.15 K), T_{R} is the room temperature (ca. 293.15 K), and P_{S} is the standard pressure for gases (1 atm). The use of a 70/30 H₂:CO₂ mixture in the batch cultures, instead of a stoichiometric mixture 80/20, was to avoid the formation of pressures lower than atmospheric in the Balch tubes and prevent potential air in-leakage into the tubes.

16S rRNA Gene Sequencing and Alignment

DNA extraction was performed via crude alkaline lysis with 0.2 M NaOH (1% SDS, 30 seconds of incubation) from a pellet of overnight grown M. wolfeii BSEL1 cells; the DNA extract was directly used in PCR. The 16S rRNA gene amplification was performed using two different sets of PCR primers: 40F (5′-GAT TAA GCC ATG CAA GTC GAA CGA-3′) (SEQ ID NO: 1), 765R (5′-CAT CGT TTA CGG CCA GGA CTA C-3) (SEQ ID NO: 2), 450F (5′-CTT CTG GAA TAA GGG CTG GGC A-3′) (SEQ ID NO: 3), 1430R (5′-CTC CTC AAA GAA CCC AGA TTC GAC-3′) (SEQ ID NO: 4). Following amplification, enzymatic cleanup was performed using Exonuclease I—Shrimp alkaline phosphatase. The amplicons were dye-terminator Sanger sequenced in a 3730xl DNA Analyzer (Applied Biosystems) according to the manufacturer instructions. The primer sequences were trimmed from the raw chromatograms. The cured forward sequences were further validated against their reverse complementary homologs, 100% matches were obtained between forward and reverse sequences. The validated sequences were initially compared with the NCBI library “16S Microbial Sequences” using BLAST 2.7.1+ for pre-identification. The evolutionary history of M. wolfeii BSEL1 was inferred using the maximum likelihood method and Tamura-Nei model. This analysis involved the 15 nucleotide sequences of the known species in the Methanothermobacter genus.

There was a total of 1,507 positions in the final data set. The evolutionary analysis was conducted in MEGA11.

### Whole-Genome Mapping

Genomic DNA was extracted from a frozen stock of M. wolfeii BSEL1 using bead-beating for cell lysis and a silica column for genomic DNA purification. DNA sequencing libraries were prepared using the NEB®Next Ultra™ II DNA Library Prep Kit (PCR-free) (NEB, USA) for Illumina, following the manufacturer instructions. The genomic DNA was fragmented by acoustic shearing with a Covaris S220 instrument. Fragmented DNA was cleaned up and end repaired. Adapters were ligated after adenylation of the 3′ ends followed by enrichment by limited cycle PCR. DNA libraries were validated using a High Sensitivity D1000 ScreenTape on a TapeStation (Agilent, USA) and were quantified using a Qubit 2.0 Fluorometer.

The sequencing library was clustered onto a flow cell on a MiSeq instrument (Illumina, USA) according to the manufacturer instructions. The sample was sequenced using a 2×250 bp paired-end configuration. Image analysis and base calling were conducted by the Illumina Control Software. Raw sequencing data were converted into fastq files using Illumina bcl2fastq 2.20. One mismatch was allowed for index sequence identification.

FastQC v.0.12.1 was used to verify the quality of the sequencing data before and after quality trimming. Four trimming operations were conducted using Trimmomatic v.0.38 to remove low-quality and short reads. The trimming operations involved leading and trailing (3 bp length), as well as sliding window trimming averaging across four bases and a quality threshold of 15. Reads under 30 bp were also removed. Quality check and data curation were conducted in a Galaxy environment. The trimmed reads were then de novo assembled using SPAdes v.3.10 with a minimum contig length of 1,000 bp and SKESA v.2.4 with hash count enabled (13 contigs were assembled). Annotation was conducted using NCBI PGAP with taxonomy check enabled. Genome assembly and annotation were performed in a WSL Ubuntu 22.04.2 LTS operating system. PGAP dependencies were stored and run in a Docker container. The completeness of M. wolfeii BSEL1 genome was estimated using PGAP against the default model organism for the Methanothermobacter genus. The circular genomic map was built in Proksee.ca.

### Growth Assays and Modeling

The growth curves in the various conditions were obtained by monitoring the optical density (λ=590 nm) within the Balch tubes using a spectrophotometer TS Genesys 20 (Thermo Scientific™, USA) equipped with a tube holder. Blanks with sterile growth medium were used to adjust the spectrophotometer to zero. During the test with YE and vitamins, the spectrophotometer was adjusted to zero using a specific blank for each treatment to avoid the loss of optical definition while using mediums with various compositions. Linear correlation was established between the optical density readings and dry biomass concentration x (R²=0.9912). x was measured using gravimetry. The logistic population growth model (equation 2) was fitted to the biomass concentration measurements during exponential phase of M. wolfeii BSEL1. The specific growth rate was equal to the slope of the linearized growth curve (equation 3). The duplication time td in the various conditions was calculated using Eq. 4. Prior to every assay, M. wolfeii BSEL1 wild type was cultivated in a serum vial until exponential phase was reached to be transferred into the Balch tubes. The inoculation order during every assay was randomized.

$${\int }_{{x}_{0}}^{x} \frac{dx}{x} = μ {\int }_{t 0}^{t} dt \qquad ( 2 )$$

$$ln \frac{x}{{x}_{0}} = μ t \qquad ( 3 )$$

$${t}_{d} = \frac{ln 2}{μ} \qquad ( 4 )$$

### Microscopy

Phase contrast and fluorescent microscopy were performed on a DMI6000B inverted microscope (Leica, Germany). For fluorescence microscopy, the cells were observed immediately after centrifugation at 4,000 g for 10 min. The microscope was equipped with a filter cube 19001-AT (Chroma, USA) which had a 420/40 excitation filter (T: 420±20 nm) and a 465 nm long pass barrier/emission filter (T: 465 nm+), corresponding with the excitation and emission wavelengths of the F420 cofactor, respectively.

### Gas Analysis

Gas composition was measured in a mass spectrometer UGA 100 (SRS, USA) equipped with an ionizer, a quadrupole, and a Faraday cup detector. The peaks at 2, 15, and 44 atomic mass units (amu) were used for H₂, CH₄, and CO₂ quantification, respectively. The signal at 16 amu was not appropriate for CH₄ analysis as it overlaps with the response to an ionic fragment of CO₂ in the mass spectrum. Standard curves were developed using standard gases (Oxarc, USA) diluted in N₂. The calibration gas mixtures for this purpose were calculated using Dalton law. The gases volumetric compositions and the mass spectrometer signals had correlation coefficients of >0.99.

### Bioreactor Used for CO₂ Fermentation

A non-stirred 8 L cylindrical bubble column reactor made of stainless steel was used to cultivate M. wolfeii BSEL1 with a constant pressure of 134.31 kPa. The pressure was analogically controlled by a pressure switch (Tameson, UK) connected in feedback loop with a solenoid valve (Parker, USA). The temperature was controlled by a temperature controller (SOLO 4848, AutomationDirect, USA) connected to a heating belt, covering the outer surface of the bioreactor, and a thermocouple installed on the lid of the bioreactor, sensing the temperature at half of the bubble column inside the bioreactor. The recirculation superficial gas velocity (2 cm/min) was powered and controlled using a peristaltic pump (Watson-Marlow 520S, USA). A carbonation stone located at the bottom of the reactor was connected to the gas distribution line. A pressurized storage tank was used to distribute the gas substrate to the reactor. The system relied on Tri-clamp, Swagelok, Luer-lock, and barbed fittings in order to maintain gas tightness. The gas substrate mimicked a biogas:H₂ mixture (26% CH₄, 13% CO₂, 61% H₂) with a H₂:CO₂ ratio equal to the stoichiometric ratio required for hydrogenotrophic methanogenesis (CO₂+4 H₂→CH₄+2 H₂O).

### Statistical Analyses

Kruskal-Wallis tests and Conover post hoc tests were conducted in R (4.4.1).

While the invention has been described in terms of its several exemplary embodiments, those skilled in the art will recognize that the invention can be practiced with modification within the spirit and scope of the appended claims. Accordingly, the present invention should not be limited to the embodiments as described above but should further include all modifications and equivalents thereof within the spirit and scope of the description provided herein.

1. An engineered strain of Methanothermobacter wolfeii deposited at the American Type Culture Collection under the number PTA-127814, and progeny and mutants thereof.

2. An M. wolfeii culture, comprising the engineered strain of claim 1 and an anaerobic culture medium.

3. The M. wolfeii culture of claim 3, wherein the culture medium is selected from at least one of a wastewater and a mineral media.

4. The M. wolfeii culture of claim 3, further comprising a supply of carbon dioxide gas and a supply of hydrogen gas.

5. The M. wolfeii culture claim 3, wherein the culture medium does not include organic macronutrients.

6. The M. wolfeii culture of claim 3, wherein the culture is maintained at a pH of 8 to 8.5.

7. The M. wolfeii culture of claim 3, wherein the culture is maintained at a temperature of 62-66° C.

8. The M. wolfeii culture of claim 3, further comprising resazurin as a redox indicator and cysteine and/or Na₂S as a reducing agent(s).

9. The M. wolfeii culture of claim 3, wherein the culture does not comprise tungsten.

10. A method of biogas production, comprising: culturing an engineered strain of claim 1 with a supply of carbon dioxide gas and a supply of hydrogen gas under anaerobic conditions.

11. A method of upgrading biogas to renewable natural gas, comprising: culturing an engineered strain of claim 1 with a supply of carbon dioxide gas and a supply of hydrogen gas under anaerobic conditions.

12. A method of carbon sequestration, comprising: culturing an engineered strain of claim 1 with a supply of carbon dioxide gas and a supply of hydrogen gas under anaerobic conditions.

13. The method of claim 13, wherein the carbon dioxide gas is or comprises waste CO₂.

14. A method of producing methane, comprising:
culturing an engineered strain of claim 1 in wastewater with a supply of carbon dioxide gas and a supply of hydrogen gas under anaerobic conditions; and
recovering methane produced from the culturing step.

15. A culture medium comprising means for converting CO₂ and H₂ to methane in a minimal media, wherein the means is the engineered strain of claim 1.

16. The culture medium of claim 16, which does not comprise tungsten.

17. An engineered strain of Methanothermobacter wolfeii having a formylmethanofuran dehydrogenase F subunit (fwdF) with serine, aspartic acid and isoleucine at positions 17, 69, and 228, respectively, and a D subunit (fwdD) with valine at position 46.
